(19) United States
(12) Patent Application Publication
(10) Pub. No.: US 2003/0157037 A1
(43) Pub. Date: Aug. 21, 2003

(54) Use of aryl oximes for the prophylaxis and/or treatment of erythema formation and/or inflammatory reaction of the skin

(76) Inventors: Joachim Bunger, Gross-Umstadt (DE); Hansjurgen Driller, Gross-Umstadt (DE); Michael Schwarz, Weiterstadt (DE); Wolfgang Wohlrab, Halle (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/296,059

(22) PCT Filed: May 8, 2001
(86) PCT No.: PCT/EP01/05222

(30) Foreign Application Priority Data
May 24, 2000 (DE) 10025557.4
May 24, 2000 (DE) 10025555.8

(51) Int. Cl. A61K 31/15; A61K 31/44; A61K 7/42

(52) U.S. Cl. 424/59; 514/357; 514/640

(57) ABSTRACT

The present invention relates to the use of at least one aryl oxime of Formula (I).

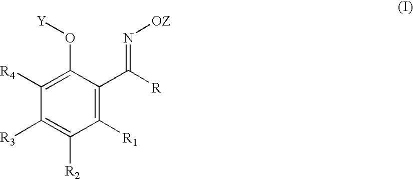

wherein:

Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl,

for the prophylaxis and/or treatment of erythema formation and/or inflammation responses of the skin.

The present invention relates to the use of at least one aryl oxime for the prophylaxis and/or treatment of erythema formation and/or inflammation responses of the skin.

Inflammations are observed in many diseases as symptoms whereby these inflammations occur, either casually or as a secondary manifestation due to pathological changes. Moreover, they may be caused by external chemical or physical noxae. An inflammation is a multi-functional event of different morphological and functional factors. These factors concern in this connection disorders in the cellular area, in the blood circulation, inflammation-induced transudation and exudation, infiltration and proliferation. Together with these disorders further changes may occur, so that inter alia spongiosis, acanthosis or parakeratosis will occur.

During the release, course and the regulation of many of these events, mediating systems are involved. Thus, lymphokinines released from sensitized T-lymphocytes are substantially involved in the cellular immune response with a large number of biological effects (Schöpf, E., Korting, G. W. [Editor] Dermatologie u. Praxis Vol. 1, Thieme: Stuttgart, N.Y. [1980]). Furthermore, in this connection, the effect of kinines, activated complementing factors, lysosomal enzymes, cyclic nucleotides and various epidermal factors are known. Prostaglandins and leukotrienes play a particular roll. As an example of a prostaglandin effect, a chemotactic effect on leukotrienes is known which decreases the vessel permeability chronologically after the kinines. Leukotrienes, on the other hand, act chemotactically on the granulocytes and influence the contractability and permeability of the vessels.

Apart from the histamine release, UV-B erythema is mediated by the arachidonic acid cascade, whereby an increased cyclo-oxygenase mediated prostaglandin synthesis, in particular, of PGE₂ and PGF₂ occurs. The lipoxygenase pathway via 5-HPETE and LTA4 leads to the essential elements of the inflammation, such as cellular infiltration of the inflamed tissue and oedema formation (review in: Gallin, J., Goldstein, I. M., Snyderman, R., [Editor], Inflammation: Basic principles and clinical correlates, New York, Raven Press [1988]).

Different active agents for the treatment of inflammations are known. Corticosteroids have the greatest importance for the treatment of the mechanisms mentioned above, which lead to different skin diseases. Weak to medium strong corticosteroids, mainly non-fluorinated derivatives of hydrocortisone, are mainly employed for the therapy of inflammatory, allergic and pruriginous skin diseases. However, during a treatment with corticosteroids undesired side effects occur depending on the employed active agent, the type and duration of the treatment, whereby these side effects must be observed and taken into consideration by any means when using these substances (Review: Symposium in Topical Corticosteroids. In: Drugs Vol. 36, 5 [1988]). For these reasons, it is preferred to use non-steroidal anti-inflammatory active agents, whereby the therapeutic efficiency of the substances known to date is, however, very limited and in most cases below that of hydrocortisone. This concerns active agents such as salicylic acid, acetyl salicylic acid, bufexamac, bendazac, phenylbutazone, oxyphenbutazone, diflumidone, indometacine and, partially also, anti-histamines (Gloor, M., Pharmakologie dermatologischer Externa. Springer Verlag Berlin Heidelberg New York [1982]).

UV irradiation belongs to the physical noxae and has positive as well as negative effects on the human skin and the whole organism. In a suitable dose, the radiation by sunlight increases the well-being and productivity of the organism. Vitamin D synthesis is stimulated and, as a result of the radiation, there is also a development of the desired tanning or pigmentation of the skin. The pigmentation is part of the skin's own protection which is based on a large number of mechanisms. In combination with the skin's own protection, there are of importance apart from the pigmentation, in particular the thickening of the horny layer (“Lichtschwiele”), the dark repair system (enzymatic DNA repair), the redox systems for the control of radical reactions and the synthesis of urocanic acid (P. Finkel, “Lichtschutzmittel” in W. Umbach, Kosmetik, 2. Edition, 1995, 147-163, Georg Thieme Verlag, Stuttgart).

An excessive radiation by sun leads to both acute skin damages such as sunburn, and also chronic changes such as skin aging or skin cancer. The sunburn (erythema solare) is developed mainly as a cause of UV-B irradiation. UV-A irradiation, on the other hand, has a comparatively low influence on its formation. The sunburn can occur in the form of a slight reddening to a strong burn with vesicle formation. Because these effects appear 4 to 6 hours after the irradiation at the earliest, it is too late for counter-measures. A sunburn is a sign for acute skin damages which may be of relevance for chronic changes of the skin. Several sunburns, in particular during the childhood, considerably increase the risk of skin cancer. Causes for this are damages, specifically of the nucleic acids of human skin cells and an imperfect repair of the damaged deoxyribonucleic acid in the nucleus of the cell as well as probably the immuno-suppressive effect of the UV irradiation, i.e. the weakening of the immune response by UV irradiation. The excessive UV-A and UV-B exposure contributes to the skin aging or photo-aging, e.g. by means of structural changes of the connective tissue (actinic elastosis). The excessive UV-B exposure is the essential cause for chronic skin changes.

Due to the change in spending one's leisure time, such as extensive sun bathing or long distance travel into countries with a strong sun radiation, the dangers of a UV damage of the skin cells have heavily increased in the last years, which in turn has resulted in an increase of the risk of skin cancer (P. Finkel, “Lichtschutzmittel” in W. Umbach, Kosmetik, 2. Edition, 1995, 147-163, Georg Thieme Verlag, Stuttgart). A particular potential danger is long distance travel into countries with a strong sun radiation in winter. The winter skin, e.g. of northern Europeans, is little pigmented and not protected against a strong sun exposure in tropical regions near the equator with a long duration of sunshine per day. In addition, the risk of skin cancer has particularly increased in recent times because of a longer life expectation of the people and because of an increased UV radiation on the surface of the earth caused by a decrease of the ozone layer.

Conventionally, commercially available UV filters which are incorporated into formulations, such as sun lotions or oils, are used for the protection of the skin against UV irradiation. However, when used too late or in a too low dose, skin reddening and even sunburn can occur with these skin protections.

PUVA therapy is a special type of UV irradiation. The abbreviation PUVA stands for psoralenes plus UV-A and means a photo-activated chemotherapy for the treatment of psoriasis. The PUVA therapy is also used in connection with vitiligo, cutaneous T-cell lymphoma, mastocytosis, sclerodermia circumscripta, granuloma anulare, polymorphous photo-dermatosis (prophylactically), prurigo, lichen ruber planus, solar urticaria, graft-versus-host reaction and acinic reticuloid. The part to be treated is selectively irradiated with UV-A rays (320 to 400 nm). Before the UV-A rays take effect, a. photo-sensitizing substance, e.g. 8- or 5-methoxypsoralene is locally or orally applied. Due to the linking of DNA strands, the cell division is hindered.

The disadvantage of PUVA therapy is an increased risk of skin cancer in the long term treatment with high accumulative doses. Due to the increased UV exposure within the PUVA therapy, there is therefore the danger that the skin is damaged as a result of the long period treatment.

It is therefore the object of the present invention to provide a means suitable for the prophylaxis and/or treatment of erythema formation and/or inflammation responses of the skin and which is particularly effective for the prophylaxis and/or treatment of inflammation responses of the skin caused by physical or chemical noxae and/or foreign organisms and which avoids or minimizes the development of erythema formation and inflammation responses of the skin during radiation with UV light, so that in particular, during the PUVA therapy, an increase of the irradiation intensity can be achieved without the risk of a skin damage or even an increased risk of skin cancer.

This object is achieved with the use of at least one aryl oxime of Formula (I).

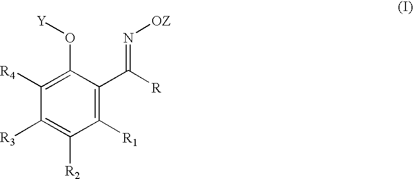

wherein,

Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H. C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl,

for the prophylaxis and/or treatment of erythema formation and/or inflammation responses of the skin.

Surprisingly, it was found that aryl oximes of Formula (I) can be used beforehand with clinically healthy patients as regards their skin and/or in the interval without clinical symptoms, for the suppression of an erythema formation or an inflammation response of the skin.

Furthermore, it was surprisingly found that aryl oximes of Formula (I) effectively control also skin damages which occur due to physical or chemical noxae and/or foreign organisms and that the active agent can also be applied prophylactically to the skin in order to serve as an effective protection from inflammation responses of the skin caused by physical or chemical noxae and/or foreign organisms.

The aryl oxime used in accordance with the present invention is represented by the Formula (I):

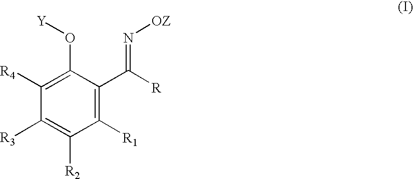

wherein,

Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl.

Alkyl, alkenyl, carboxy alkyl, carboxy alkenyl, alkanoyl, cycloalkyl, alkoxy, aryl, aryloxy, and aralkyl can be unsubstituted or substituted. Suitable substituents of these groups are preferably alkyl, alkoxy, alkenyl, aryl, aryloxy, aralkyl, heteroaryl, heteroaralkyl, hydroxy, carboxy, carboxy alkyl, dialkyl amine, sulfonyl and combinations thereof.

Alkyl is respectively straight chain or branched alkyl and is therefore preferably methyl, ethyl, propyl, isopropyl, butyl, isobutyl, t-butyl, pentyl, hexyl, heptyl, octyl, nonyl, decyl, undecyl, dodecyl, tridecyl, tetradecyl, pentadecyl, hexadecyl, heptadecyl and octadecyl.

Alkenyl means that in the specified alkylene moiety one or more double bonds may be present.

Aryl is an aromatic C₆₋₂₀ hydrocarbon residue and is preferably phenyl.

Aralkyl is an alkyl group substituted with aryl and has preferably the meaning of benzyl or phenethyl.

Cycloalkyl is a cyclic alkyl group and is preferably cyclopropyl, cyclobutyl, cyclopentyl, cyclohexyl, cycloheptyl or cyclooctyl.

Heteroaryl is an aromatic ring with heteroatoms, preferably a nitrogen-containing ring, such as pyridyl or pyrimidyl.

Heteroaralkyl means a alkyl group substituted with heteroaryl and is preferably pyridyl methyl and pyrimidyl methyl.

Suitable condensed systems are preferably the residues naphthyl, benzofuryl, quinolinyl, indolyl or cinnolinyl.

Dialkyl amine means NR₅R₆ wherein R₅ and R₆ may be the same or different, and C₁₋₁₂ alkyl.

Z and Y are preferably independently from each other a hydrogen atom, a C₁₋₆ alkyl group which can have at least one substituent selected from —OH, —COOH, —SO₃H or NR₅R₆, an alkanoyl group represented by —C(O)R₇ wherein R₇ is a C₁₋₆ alkyl group which may have at least one substituent selected from —OH, —COOH or —SO₃H, or a CONHR₈ group wherein R₈ is a C₆₋₂₀ aryl group. Particularly preferably Z and Y are independently from each other a hydrogen atom, —(CH₂)₁₋₆COOH, —CH₂CH(OH)CH₂OH, —(CH₂)₁₋₆SO₃H, —(CH₂)₁₋₆NR₅R₆ or C(O)(CH₂)₁₋₆COOH.

Substituent R is preferably a C₁₋₁₂ alkyl group, particularly preferred are C₁₋₅ and C₁₁ alkyl groups.

Substituent R₁ is preferably a hydrogen atom or a chlorine atom.

Substituent R₂ is preferably a hydrogen atom or a chlorine atom or a C₁₋₆ alkyl group. Particularly preferred are a hydrogen atom, a chlorine atom and a methyl group.

Substituent R₃ is preferably a hydrogen atom or a C₁₋₆ alkyl group, a C,-6 alkoxy group, a O-cyclohexyl group or a benzyl group.

Substituent R₄ is preferably a hydrogen atom or a chlorine atom.

R₁, R₂, R₃ and R₄ may be preferably substituted, if possible, with —OH, —COOH, —SO₃H or —NR₅R₆ to increase, e.g. the water solubility.

Preferred examples of the aryl oxime used in accordance with the present invention include:

4-methyl-2-hydroxy-caprophenone oxime, 5-methyl-2-hydroxy-caprophenone oxime, 5-methyl-2-hydroxy-caprophenone (N-phenylcarbamoyl) oxime, 5-methyl-2-hydroxy-laurophenone oxime (2-hydroxy-5-methyl-laurophenone oxime), 3-chloro-2-hydroxy-caprophenone oxime, 4-pentoxy-2-hydroxy-acetophenone oxime, 4-decyloxy-2-hydroxy-acetophenone oxime, 4-benzyloxy-2-hydroxy-acetophenone oxime, 4-decyloxy-2-hydroxy-propiophenone oxime, 4-butoxy-5-n-hexyl-2-hydoxy-acetophenone oxime, 4-pentoxy-2-hydroxy-caprophenone oxime, 4-decyloxy-2-hydroxy-caprophenone oxime, 4-octyloxy-2-hydroxy-laurophenone oxime, 4-cyclohexyl-oxy-2-hydroxy-propiophenone oxime, 5-chloro-2-hydroxy-caprophenone oxime, 3-chloro-2-hydroxy-laurophenone oxime, 5-chloro-2-hydroxy-laurophenone oxime, 4-butoxy-2-hydroxy-acetophenone oxime, 4-dodecyloxy-2-hydroxy-propiophenone oxime, 4-hexadecyloxy-2-hdroxy-acetophenone oxime, 4-octadecyloxy-2-hydroxy-acetophenone oxime, 4-decyloxy-2-hydroxy-laurophenone oxime, as well as the following oxime derivatives of 2-hydroxy-5-methyl-laurophenone oxime:

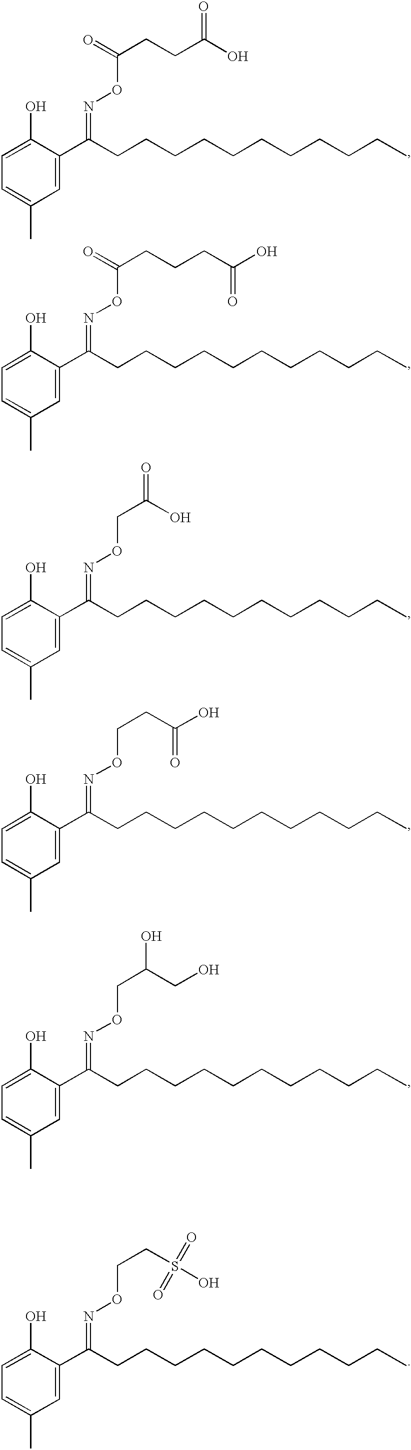

As well as the mixtures of these compounds

Particularly preferred are 2-hydroxy-5-methyl laurophenone oxime as well as its oxime derivatives mentioned before.

The aryl oximes of Formula (I) are used in accordance with the present invention for the prophylaxis and/or treatment of inflammation responses of the skin caused by physical or chemical noxae and/or foreign organisms. Physical noxae include electromagnetic rays or mechanical irritations. Examples of chemical noxae include agrochemicals, medicaments, insecticides, solvents and dusts. The term “foreign organisms” includes skin infections with different bacteria, viruses, skin pathogenic fungi and parasites.

It is preferred that at least one aryl oxime of Formula (I) is used in accordance with the present invention for the prophylaxis and/or treatment of inflammation responses of the skin whereby the physical noxa is UV irradiation.

Furthermore, it is preferred that at least one aryl oxime of Formula (I) is used in accordance with the present invention for the prophylaxis and/or treatment of inflammation responses of the skin in the PUVA therapy as a special type of UV irradiation.

The aryl oximes of Formula (I) are used in accordance with the present invention typically in the form of a topical composition.

At least one aryl oxime of Formula (I) is used in accordance with the present invention in a topical composition in a sufficient amount in order to be suitable for a prophylactic use. Typically, at least one 1-(2-hydroxyaryl)-alkan-1-one oxime of Formula (I) is used in the topical composition in an amount of 0.005 to 5 wt. %, preferably 0.02 to 2 wt. %, more preferably 0.05 to 1.5 wt. %.

The preparation of the topical composition is effected by bringing at least one of the compounds used in accordance with the present invention, if necessary together with auxiliaries and/or carriers, into a suitable type of a formulation. The auxiliaries and carriers are derived from the group of carrier agents, preservatives and other typical auxiliary agents.

The topical composition on the basis of at least one compound used in accordance with the present invention, is prophylactically applied externally on the skin or the skin adnexa.

Forms of using the composition include, e.g. solutions, suspensions, emulsions, pastes, ointments, gels, creams, lotions, sticks, powders, soaps, surfactant containing cleaning preparations, oils and sprays. In addition to the at least one compound used in accordance with the present invention, any usual carriers, auxiliary and, if necessary further active agents, will be added to the composition.

Preferred auxiliaries are derived from the group of preservatives, antioxidants, stabilizers, solubilizers, vitamins, coloring agents and agents to improve the odor. Ointments, pastes, creams and gels may contain, in addition to at least one compound used in accordance with the present invention, the usual carriers, e.g. animal and vegetable fats, waxes, paraffin, starch, tragacanth, cellulose derivatives, polyethylene glycol, silicones, bentonites, silica, talcum and zinc oxide, xanthane gum, glycerin, carboxypolymethylene or mixtures of these compounds.

Powders and spray may contain, in addition to at least compound used in accordance with the present invention, the usual carriers, e.g. milk sugar, talcum, silica, aluminum hydroxide, calcium silicate and polyamide powder or mixtures of these compounds. Sprays may additionally contain the usual propellants, e.g. chlorofluorinated hydrocarbons, propane/butane or dimethyl ether.

Solvents and emulsions may contain, in addition to at least one compound used in accordance with the present invention, the usual carriers, such as solvents, solubilizing agents and emulsifiers, e.g. water, ethanol, isopropanol, ethyl carbonate, ethyl acetate, benzyl alcohol, benzyl benzoate, propylene glycol, 1,3-butyl glycol, oils, in particular, cottonseed oil, peanut oil, maizegerm oil, olive oil, castor oil and sesame oil, esters of glycerin fatty acids, polyethylene glycol, xanthane gum, glycerin, carboxypolymethylene and fatty acid esters of sorbitan or mixtures of these compounds.

Suspensions may contain, in addition to at least one compound used in accordance with the present invention, the usual carriers, such as liquid diluents, e.g. water, ethanol or propylene glycol, suspending agents, e.g. ethoxylated isostearyl alcohols, polyoxyethylene sorbitan esters and polyoxyethyl sorbitan esters, microcrystalline cellulose, aluminum metahydroxide, bentonite, agar-agar gum and tragacanth, xanthane gum, glycerin, carboxypolymethylene or mixtures of these compounds.

Soaps may contain, in addition to at least compound used in accordance with the present invention, the usual carriers, e.g. alkaline salts of fatty acids, salts of fatty acid semi-esters, fatty acid protein hydrolysates, isothionates, lanolin, fatty alcohol, vegetable oils, vegetable extracts, glycerin, sugar or mixtures of these substances.

Surfactant containing cleaning products may contain, in addition to at least one compound used in accordance with the present invention, the usual carriers such as salts of fatty alcohol sulphates, fatty alcohol ether sulphates, sulphosuccinic acid semi-esters, fatty acid protein hydrolysates, isothionates, imidazolinium derivatives, methyl taurates, sarcosinates, fatty acid amide ether sulphates, alkyl amidobetaines, fatty alcohols, fatty acid glycerides, fatty acid diethanol amides, vegetable and synthetic oils, lanolin derivatives, ethoxylated glycerin fatty acid esters or mixtures of these compounds.

Facial and body oils may contain, in addition to at least one compound used in accordance with the present invention, the usual carriers such as synthetic oils, such as fatty acid esters, fatty alcohols, silicone oils, natural oils such as vegetable oils and oily plant extracts, paraffin oils, lanolin oils or mixtures of these compounds.

Further typical cosmetic forms are also lipsticks, lip caring sticks, mascara, eye liners, eye shadows, rouge, make-up in the form of powder, emulsion and wax as well as preparations for sun protection, pre-sun and after sun preparations.

It is particularly preferred that apart from the one or more compounds used in accordance with the present invention, there is used in addition, at least one ester as an emulsifier, the carboxylic acid residue of which is derived from C₅ to C₁₆ acids and the hydroxyl residue of which is derived from monomers, dimers or trimers of lactic acid or one of its salts or a polyglycerin of 2 to 10 molecules glycerin whereby per mole of polyglycerin 1 to 3 moles of carboxylic acid are present. This emulsifier serves the purpose to effect an improved stability of the compound used in accordance with the present invention.

The carboxylic acid residue of these esters is derived from the C₅-C₁₆ acids, preferably C₈-₁₂ acids. The carbon chain of the carboxylic acid residue can be saturated or partially unsaturated. Preferred examples of the carboxylic acid residue include hexanoic acid, caprylic acid, capric acid, lauric acid, myristic acid, palmitic acid, stearic acid, behenic acid, oleic acid, linoleic acid and mixtures thereof, for example coconut fatty acid (the carboxylic acid residues of which are characterized by “cocoyl”) which is a mixture of the aforementioned fatty acids.

The hydroxy residue of the ester can be derived from monomers, dimers or trimers of lactic acid or one of its salts. Preferably a monomer or dimer of lactic acid is employed. It is furthermore preferred that the lactic acid is used in the form of its salt, i.e. as the lactate.

Particularly preferred are alkali metal and alkaline earth metal salts, whereby sodium salts are particularly mentioned. Moreover, the hydroxy residue of the ester can be derived from a polyglycerin of 2 to 10 molecules of glycerin. In this case 1 to 3 moles of carboxylic acid are present per mole of polyglycerin. Particularly preferred 2 to 3 moles of carboxylic acid are present per mole of polyglycerin.

Typical examples of this emulsifier include dispersing auxiliaries as mentioned in DE-A-197 22 405, column 2, lines 38 to 56, as well as in the examples. Preferred are polyglycerin 10-tricaprylate, polyglycerin 10-trilaurate, polyglycerin 2-oleate, sodium lauryl lactate, sodium cocoyl lactate, capric/caprylic acid triglyceride and mixtures thereof. Particularly preferred are polyglycerin 2-oleate and sodium cocoyl lactate.

Typically, this emulsifier is used in the topical composition used in the present invention in an amount of 0.5 to 30 wt. %, preferably 0.5 to 20 wt %, more preferably 1 to 10 wt. %.

In order to ensure the stability of the topical composition used in the present invention and the aryl oximes of Formula (I) contained therein, there is used preferably furthermore at least one co-emulsifier selected from glycerin and sorbitan ester derivatives as well as cetearyl alcohol and ester derivatives thereof and mixtures of these substances. The glycerin, sorbitan and cetearyl ester derivatives are typically derived from esters whereby the carboxylic acid residues of which are derived from C₅₋₁₆ acids, the carbon chains of which are saturated or partially unsaturated. Particularly preferred of these are glycerin stearate, sorbitan stearate, sorbitan isostearate, sorbitan diisostearate, sorbitan dioleate, sorbitan distearate, sorbitan laurate, sorbitan palmitate, sorbitan sesquiisostearate, sorbitan sesquioleate, sorbitan triisostearate, sorbitan trioleate, sorbitan tristearate, cetearyl octanoate, cetearyl palmitate, cetearyl isononanoate and mixtures thereof.

Typically, this co-emulsifier is used in the topical composition used in the present invention in an amount of 0.1 to 40 wt. %, preferably 0.5 to 15 wt. %, more preferably 1 to 10 wt. %.

It is preferred that, in addition, at least one lipophilic solvent is present in order to further improve the solubility of the active agent in the composition of the present invention. Typical lipophilic solvents suitable for a topical formulation include dimethicone, cyclomethicone, mineral oil, isostearyl isostearate, octyl palmitate, propylene glycol/dicaprate/dicaprylate, C₁₂₋₁₅ alkyl benzoate, octyl decanol, ether derivatives of cetyl alcohol such as Ceteth-1, Ceteth-2, Ceteth-3, Ceteth4, Ceteth-5, Ceteth-6 and Ceteth-10, ethylbutylacetyl aminopropionate, ethanol, isopropanol, isopropyl myristate, and mixtures thereof. Of these ethylbutyl acetyl aminopropionate, ethanol, isopropanol, isopropyl myristate and mixtures thereof are particularly preferred.

The lipophilic solvent is typically used in the topical composition used in the present invention in an amount of 0.1 to 20 wt. %, more preferably 0.3 to 17 wt. %.

Preferably, apart from the one or more compounds used in accordance with the present invention, an antioxidant is used. The antioxidants serve as a protection from cell damage caused by radicals.

Antioxidants known from the literature in the respective field can be used in accordance with the present invention, for example flavonoides, coumaranones, amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoides, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthio uracil and other thiols (e.g. thioredoxin, glutathione, cysteine, cystine, cystamine as well as glycosyl, N-acetyl, methyl, ethyl, propyl, amyl, butyl, and lauryl, palmitoyl, oleyl, γ-linoleyl, cholesteryl and glyceryl esters of these) as well as salts of these, diaurylthiodipropionate, distearylthiodipropionate, thiodipropinoic acid and derivatives (esters, ethers, peptides, lipids, nucleotides, nucleosides and salts) of these as well as sulfoximine compounds (e.g. buthionine sulfoximines, homocysteine sulfoximine, buthionine sulfones, penta-, hexa-, heptathionine sulfoximine), furthermore (metal) chelating agents (e.g. α-hydroxy fatty acids, palmitic acid, phytic acid, lactoferrin), α-hydroxy acids (e.g. citric acid, lactic acid, malic acid), humic acid, cholic acid, cholic extracts, bilirubin, biliverdin, EDTA, EGTA and derivatives thereof, unsaturated fatty acids and derivatives thereof, vitamin C and derivatives (e.g. ascorbyl palmitate, magnesium ascorbyl phosphate, ascorbyl acetate) as well as coniferyl benzoate of the benzoic resin, rutinic acid and derivatives thereof, α-glycosyl rutin, ferulic acid, furfurylidene glucitol, carnosine, butylhydroxy toluene (BHT), butylhydroxy anisole, nordohydroguaiaretic acid, trihydroxybutyrophenon, uric acid and derivatives thereof, mannose and derivatives thereof, zinc and derivatives e.g. ZnO, ZnSO₄), selenium and derivatives thereof (e.g. selenium methionine), stilbenes and derivatives thereof (e.g. stilbene oxide, trans-stilbene oxide) and melatonin.

Mixtures of antioxidants are also suitable. Known and commercially available mixtures are, for example, mixtures containing, as active ingredients lecithin, L-(+)-ascorbyl palmitate and citric acid (e.g. Oxynex® AP), natural tocopherols, L-(+)-ascorbyl palmitate, L-(+)-ascorbic acid and citric acid (e.g. Oxynex® K LIQUID), tocopherol extracts from natural sources, L-(+)-ascorbyl palmitate, L-(+)-ascorbic acid and citric acid (e.g. Oxynex® L LIQUID), DL-α-tocopherol, L-(+)-ascorbyl palmitate, citric acid and lecithin (e.g. Oxynex® LM) or butylhydroxy toluene (BHT), L-(+)-ascorbyl palmitate and citric acid (e.g. Oxynex® 2004).

In a preferred embodiment of the invention butylhydroxy toluene is used as an antioxidant.

In another preferred embodiment one or more compounds selected from flavonoides and/or coumaranones are used as the antioxidant.

Flavonoides are understood as being the glycosides of flavanones, flavones, 3-hydroxyflavones (=flavanoles), aurones, isoflavones and rotenoides (Römpp Chemie Lexikon, Vol. 9, 1993). Within the scope of the present invention, however, also the aglycones, i.e. the sugar free components, and the derivatives of the flavonoides and the aglycones are embraced. Within the scope of the present invention coumaranones embrace also the derivatives thereof.

Preferred flavonoides are derived from flavanones, flavones, 3-hydroxyflavones, aurones and isoflavones, in particular flavanones, flavones, 3-hydroxyflavones and aurones.

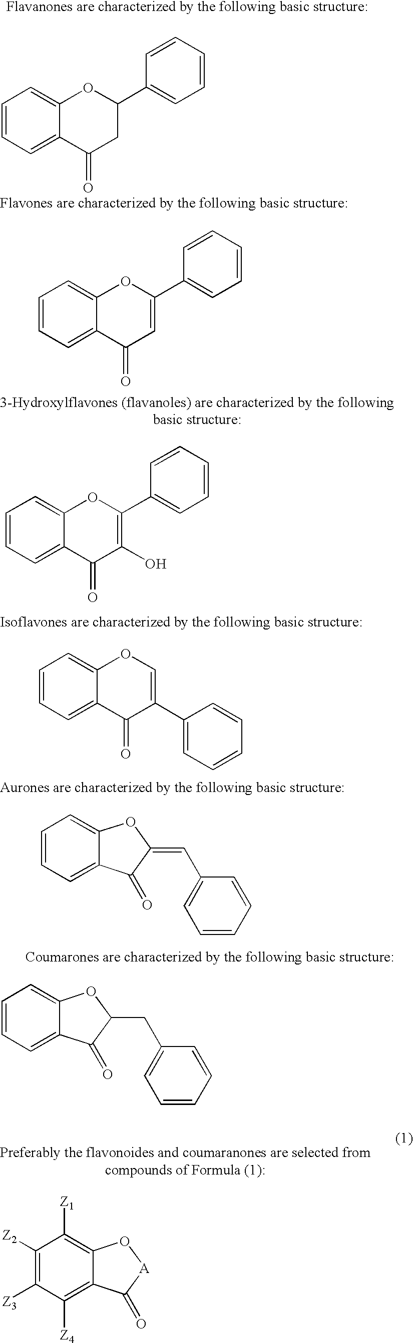

wherein

Z₁ to Z₄ each represent independently from each other, H, OH, alkoxy, hydroxyalkoxy, mono- or oligoglycoside residues, whereby the alkoxy and hydroxyalkoxy groups can be branched or unbranched and may have 1 to 18 C-atoms and wherein on the hydroxy groups of the above mentioned residues, sulphate or phosphate may also be bonded,

A is selected from the group consisting of the substructures (1A), (1B) and (1C)

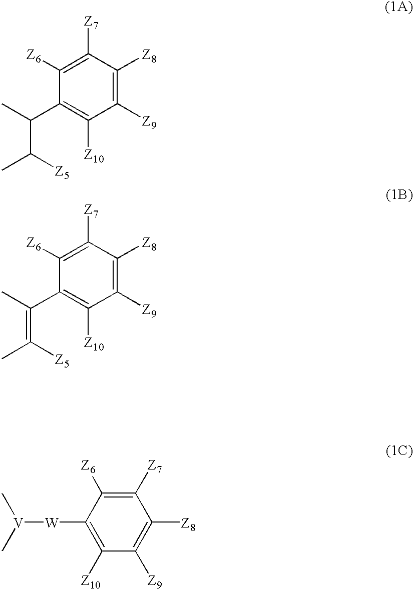

Z₅ represents H, OH, or OR,

R represents a mono- or oligoglycoside residue,

Z₆ to Z₁₀ have the meaning of the residues Z₁ to Z₄ and

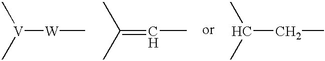

The alkoxy groups are preferably linear and have 1 to 12, preferably 1 to 8 C-atoms. These groups correspond, therefore, to Formula —O—(CH₂)ₘ—H, wherein m represents 1,2,3,4,5,6,7 or 8 and in particular 1 to 5.

The hydroxyalkoxy groups are preferably linear and have 2 to 12, preferably 2 to 8 C-atoms. These groups represent, therefore, Formula —O—(CH₂)ₙ—H wherein n represents 2,3,4,5,6,7 or 8, preferably 2 to 5 and in particular preferably 2.

The mono- and oligoglycoside residues are preferably made up from 1 to 3 glycoside units. Preferably these units are selected from the group of hexosyl residues, in particular rhamnosyl residues and glycosyl residues. However, other hexosyl residues, for example allosyl, altrosyl, galatosyl, gulosyl, idosyl, mannosyl and talosyl can be used advantageously under the circumstances. Moreover, it can be advantageous for the invention to use pentosyl residues.

In a preferred embodiment the substituents have the following meaning:

| | |
|---|---|
| Z₁ and Z₃ | the meaning H, |
| Z₂ and Z₄ | a different meaning than H, in particular they represent |
| | OH, methoxy, ethoxy or 2-hydroxyethoxy, |
| 4 | the meaning H, OH or a glycoside residue made up of 1 |
| | to 3, preferably 1 or 2 glycoside units. |
| Z₆, Z₉ and Z₁₀ | represent the meaning H, and |
| Z₇ and Z₈ | a different meaning than H, in particular they represent |
| | OH, methoxy, ethoxy or 2-hydroxyethoxy. |

In another preferred embodiment, in particular when the water solubility of the flavonides and coumaranones should be increased, a sulphate or phosphate group is bonded to the hydroxy groups. Suitable counter-ions are, for example, ions of alkali metals or alkaline earth metals, wherein these are, e.g. selected from sodium or potassium.

In another preferred embodiment the flavonoides are selected from the following compounds: 4,6,3′,4′-tetrahydroxyaurone, quercetin, rutin, isoquercetin, anthocyanidin (cyanidin), eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin) as well as sulphates and phosphates thereof.

From the flavonoides, rutin and troxerutin are particularly preferred. Especially preferred is troxerutin.

From the coumaranones, 4,6,3′,4′-tetrahydroxybenzyl coumaranone-3 is preferred.

The antioxidants are typically employed in the topical composition used in accordance with the present invention in an amount of 0.001 to 5 wt. %, preferably 0.5 to 5 wt. %.

As UV protection, there can be used preferably, apart from one or more compounds used in accordance with the present invention, at least one UV filter. In the UV therapy, such as the PUVA therapy, there are of course no UV filters employed. In accordance with the present invention, there can be used UV filters known from the literature in the respective fields. Usual amounts of the UV filters which can be used in accordance with the present invention are 0.05 to 30 wt. %, preferably 0.1 to 20 wt. %, more preferably 1 to 15 wt. %.

Suitable organic UV filters can be selected from all known UVA but also UVB filters known to the person skilled in the art. For both UV ranges there ar reliable substances known from special literature, for example:

Benzylidene camphor derivatives, such as:

3-(4′-methylbenzylidene) di-camphor (e.g. Eusolex® 6300),

3-benzylidene camphor (e.g. Mexoryl® SD),

polymers of N-{(2 and 4)-[(2-oxoborn-3-ylidene)methyl]benzyl} acrylamide (e.g. Mexoryl® SW),

N,N,N-trimethyl-4-(2-oxoborn-3-ylidene methyl)anilinium methylsulphate (e.g. Mexoryl® SK), or

α-(2-oxoborn-3-ylidene)toluene 4-sulphonic acid (e.g. Mexoryl® SL).

Benzoyl or dibenzoyl methanes, such as:

1-(4-tert-butylphenyl)-3-(4-methoxyphenyl) propane 1,3-dione (e.g. Eusolex® 9020), or

4-isopropyldibenzoyl methane (e.g. Eusolex® 8020).

Benzophenones, such as:

2-hydroxy-4-methoxy benzophenone (e.g. Eusolex® 4360), or

2-hydroxy-4-methoxy benzophenone 5-sulphonic acid and its sodium salt (e.g. Uvinul® MS-40).

Methoxy cinnamates, such as:

p-methoxy cinnamic acid 2-ethylhexylester (e.g. Eusolex® 2292),

p-methoxy cinnamic acid isopentylester, e.g. as a mixture of the isomers (e.g. Neo Heliopan® E 1000).

Salicylate derivatives, such as:

2-ethylhexyl salicylate (e.g. Eusolex® OS),

4-isopropylbenzyl salicylate (e.g. Megasol®), or

3,3,5-trimethylcyclohexyl salicylate (e.g. Eusolex® HMS).

4-aminobenzoic acid and derivatives thereof, such as:

4-aminobenzoic acid,

4-(dimethylamino)benzoic acid 2-ethylhexylester (e.g. Eusolex® 6007),

ethoxylated 4-aminobenzoic acid ethylester (e.g. Uvinul® P25).

and further substances such as:

2-cyano-3,3-diphenyl acrylic acid 2-ethylhexylester (e.g. Eusolex® OCR),

2-phenylbenzimidazole 5-sulphonic acid as well as its potassium, sodium and triethanol amine salts (e.g. Eusolex® 232),

3,3′-(1,4-phenylenedimethylene)-bis-(7,7-dimethyl-2-oxobicyclo[2.2.1]hept-1-yl methane sulphonic acid as well as its salts (e.g. Mexoryl® SX, and

2,4,6-trianilino-(p-carbo-2′-ethylhexyl-1′-oxy) 1,3,5-triazine (e.g. Uvinul® T 150).

These organic UV filters are typically employed in the topical composition used in the present invention in an amount of 0.5 to 10 wt. %, preferably 1 to 8 wt. %.

Further suitable organic UV filters are, for example:

2-(2H-benzotriazol-2-yl)-4-methyl-6-(2-methyl-3-(1,3,3,3-tetramethyl-1-(trimethylsilyloxy)disiloxanyl)propyl) phenol (e.g. Silatrizole®),

4,4′-[(6-[4-((1,1-dimethylethyl)aminocarbonyl)phenylamino] 1,3,5-triazin-2,3-diyl)diamino] bis-benzoic acid 2-ethylhexylester (e.g. Uvasorb® HEB),

α-(trimethylsilyl)-ω[trimethylsilyl)oxy]poly[oxy(dimethyl] [and approx. 6% methyl[2-[p-[2,2-bis-(ethoxycarbonyl]vinyl]phenoxy]-1-methylene ethyl] and approx. 1.5% methyl[3-[p-[2,2-bis-(ethoxycarbonyl)vinyl)phenoxy) propenyl) and 0.1 to 0.4% (methylhydrogen]silylene]] (n≈60) (e.g. Parsol® SLX),

2,2′-methylene-bis-(6-(2H-benzotriazol-2-yl) 4-(1,1,3,3-tetramethylbutyl) phenol (e.g. Tinosorb® M),

2,2′-(1,4-phenylene)-bis-1H-benzimidazole 4,6-disulphonic acid monosodium salt,

2,2′-(1,4-phenylene)-bis-1H-benzimidazole 5-disulphonic acid monosodium salt,

2,2′-(1,4-phenylene)-bis-1H-benzimidazole 5-disulphonic acid monopotassium salt, and

2,4-bis-[4-(2-ethyl-hexyloxy)-2-hydroxyl]-phenyl 6-(4-methoxyphenyl) 1,3,5-triazine (e.g. Tinosorb® S).

These organic filters are typically emloyed in the topical composition used in the present invention in an amount of 0.5 to 20 wt. %, preferably 1 to 15 wt. %.

Inorganic UV filters can be selected from the group of titanium dioxides, e.g. coated titanium dioxide (e.g. Eusolex® T-2000 or Eusolex® T-Aqua), zinc oxide (e.g. Sachtotec®), iron oxides or also cerium oxides. These inorganic UV filters are typically employed in the topical composition used in the present invention in an amount of 0.5 to 20 wt. %, preferably 2 to 10 wt. %.

Preferred UV filters are zinc oxide, titanium dioxide, 3-(4′-methylbenzylidene) dl-camphor, 1-(4-tert-butylphenyl) 3-(4-methoxyphenyl)propan-1,3-dione, 4-isopropyl dibenzoyl methane, 2-hydroxy-4-methoxybenzophenone, 4-methoxy cinnamic acid 2-ethylhexyl ester, 3,3,5-trimethylcyclohexyl salicylate, 4-(dimethylamino)benzoic acid 2-ethylhexyl ester, 2-cyano-3,3-diphenyl acrylic acid 2-ethylhexyl ester, 2-phenyl benzimidazole-5-sulphonic acid, as well as their potassium, sodium and triethanol amine salts.

Especially preferred UV filters are zinc oxide and titanium dioxide.

When titanium dioxide is used in accordance with the present invention, it is preferred that apart from titanium dioxide, in addition, one or more further UV filters selected from 3-(4′-methylbenzylidene) dl-camphor, 1-(4-tert-butylphenyl)-3-(4-methoxyphenyl)propan-1,3-dione, 4-isopropyl dibenzoyl methane, 2-hydroxy-4-methoxybenzophenone, p-methoxycinnamic acid 2-ethylhexyl ester, 3,3,5-trimethylcyclohexyl salicylate, 4-(dimethylamino)benzoic acid 2-ethylhexyl ester, 2-cyano-3,3-diphenyl acrylic acid 2-ethylhexyl ester, 2-phenyl benzimidazole 5-sulphonic acid, as well as their potassium, sodium and triethanol amine salts are employed.

It is particularly preferred that apart from titanium dioxide, in addition, the UV filters 2-hydroxy-4-methoxy benzophenone and/or methoxy cinnamic acid octyl ester are employed.

In order to improve the protection of the skin and an immuno suppression of the skin, the combination of aryl oximes with ectoin and ectoin derivatives is particularly effective.

The invention is further illustrated by means of the following examples.

### EXAMPLE 1

Analysis of the Prophylactic Effect

Materials and Methods

A 10% solution of 2-hydroxy-5-methyl-laurophenone oxime in absolute ethanol was used as the examination substance. The isotope labeling was effected with 2-hydroxy-5-methyl-[1-¹⁴C]-laurophenone oxime, specific activity: 110 MBq/g, so that 100 μl of a 10% solution included an activity of 27.46 μCi for the tests.

The analyses were carried out on human skin of operation preparations after amputation of the breast. The subcutis was removed post-operatively, the skin was cut to the size and area necessary for the test, packed in aluminum foil and stored for a short period at −20° C. The test method was carried out according to Dermatol. Mon.schr. 167 (1981) p. 277-283.

The part of the skin to be analyzed was attached with pins without tension at the corners on a synthetic fiber sieve after an area of 4 cm² was marked on the skin. 20 μl of the ¹⁴C-labeled solution of the active agent were applied onto the test area and uniformly spread over the test area. Immediately after the application, the synthetic fiber sieve was attached in a glass vessel with physiological NaCl solution, such that the NaCl solution which was continuously agitated with a magnetic stirrer was in contact with the lower area of the skin. The whole apparatus was assembled in an incubator so that during the whole period of the experiment, the temperature could be kept constant at 32° C.

The penetration measurements were carried out on two or three different operation preparations, respectively. The work-up of the skin took place 30, 300 and 1000 minutes after the application of the solution of the substance. In order to do so, the surface of the skin was first of all wiped with cotton wool and attached on a synthetic pad. Then a stencil was attached onto the test area whereby an area of 1 cm² was omitted in the stencil. On this area, there took place a layer-by-layer removal of the stratum corneum with an adhesive film using tearing, whereby each tearing was separately transferred into a sample glass.

After removal of the stratum corneum, several skin cylinders were punched out using a fast rotating punch (diameter 4 mm) and on the freezing microtome horizontal cuts were prepared. At first, 200 μm cuts were prepared up to a depth of the tissue of 200 μm whereby, as a means of simplification, a depth of skin of 160 μm was considered as being the epidermis. The remaining tissue, the dermis, was worked up completely in 40 μm cuts. As with the tearings of the stratum corneum, each cut was transferred individually into a sample glass. For the scintillation measurement, the tissue cuts were incubated each for approximately for 12 hours with 0.2 ml protosol (New England Nuclear) for solubilisation and afterwards mixed with 2 ml methanol. For the measurement in the liquid scintillation spectrophotometer, all sample glasses were charged with 10 ml scintillation liquid each (4.0 g PPO+0.1 g POPOP+1000 ml toluene). The respective quench was taken into account by means of an external standard. In order to convert cpm into dpm, calibration curves were used. Using further calibration curves, dpm was converted into μCi. Due to the knowledge of the applied amount of the substance of the specific activity, the area of the stripped part of the skin, the volume (area and layer thickness) of the histological cuts as well as their assignment to the individual skin layers, the amount of the penetrated substance could be calculated as a percentage of the applied amount or a molar concentration in the respective skin layer.

The results are shown in Table 1.

By summarizing all the activities found again in the tearings of the stratum corneum, it is possible to determine the percentage of the penetrated amount of the externally applied active agent in the stratum corneum for each penetration time. A corresponding finding is possible by the summation of the measured value of the tissue cuts up to a depth of the layer of 160 μm for the epidermis and the remaining measured values for the dermis.

It can be ascertained from the measured results shown in Table 1, that the predominant amount of the active agent penetrated into the human skin can be found in the stratum corneum. The distribution of the amount of the active agent penetrated into the stratum corneum, within the stratum corneum, is shown in Table 2. The active agent penetrates relatively rapidly into the superficial horny layers with a marked concentration drop to the lower horny layers. This ratio shifts more and more with increasing penetration duration, so that after 1000 minutes there is an almost uniformly distributed amount of the active agent over the whole stratum corneum.

This corresponds to the obtained concentrations of the active agent in the individual skin layers with increasing penetration time as shown in Table 3. As a result, only after longer penetration times, an increase in the epidermal concentration of the active agent can be found which is clearly lower in the dermal area.

On a whole, these results show that 2-hydroxy-5-methyl-laurophenone oxime forms a depot of the active agent facilitating the prophylactic use, respective care, for the protection of the skin against pro-inflammatory factors.

TABLE 1
Penetration of 2-hydroxy-5-methyl-laurophenone oxime into the
human skin after external application (in % of the applied amount)
Application: 10% 2-hydroxy-5-methyl-laurophenone oxime in
ethanol (abs.);
20 μl/4 cm² × N = 3.

| | Penetration Time [min.] | | |
|---|---|---|---|
| | 30 | 300 | 1000 |
| stratum corneum | 12.36 ± 6.74 | 18.27 ± 2.69 | 15.45 ± 1.34 |
| epidermis | 1.99 ± 1.28 | 2.00 ± 0.09 | 4.44 ± 1.51 |
| (<160 μm depth of the skin) | | | |
| dermis | 1.83 ± 0.60 | 3.94 ± 2.86 | 5.62 ± 3.17 |
| (>160 pm depth of the skin) | | | |

TABLE 2
Penetration of 2-hydroxy-5-methyl-laurophenone oxime (in % of
the applied amount) into the stratum corneum of human skin after
external application
Application: 10% 2-hydroxy-5-methyl-laurophenone oxime in
ethanol (abs.);
20 μl/4 cm² × N = 3.

Penetration Time [min.]

| | 30 | 300 | 1000 |
|---|---|---|---|
| tearings 1-5 | 9.41 ± 5.24 | 12.95 ± 1.13 | 7.04 ± 4.01 |
| tearings 6-20 | 2.94 ± 1.50 | 6.22 ± 1.57 | 8.41 ± 5.35 |

TABLE 3
Penetration of 2-hydroxy-5-methyl-laurophenone oxime into the human skin after
external application (in micromolar concentration)
Application: 10% 2-hydroxy-5-methyl-laurophenone oxime in ethanol (abs.);
20 μl/4 cm² × N = 3.

| | Penetration Time [min.] | | |
|---|---|---|---|
| | 30 | 300 | 1000 |
| stratum corneum | 60.69 ± 33.08 | 149.51 ± 22.05 | 75.86 ± 6.56 |
| (thickness \hat{=} 20 μm) | | | |
| epidermis | 1.22 ± 1.04 | 2.04 ± 0.09 | 2.72 ± 1.18 |
| (thickness \hat{=} 160 μm) | | | |
| dermis | 0.28 ± 0.09 | 1.01 ± 0.73 | 0.86 ± 0.08 |
| (thickness \hat{=} 40 μm × amounts of cuts) | | | |

### EXAMPLE 2

Analysis of UV Conditioned Inflammation Responses of the Skin

Materials and Methods

Albino guinea pigs were used as experimental animals.

A solution containing 2-hydroxy-5-methyl-laurophenone oxime was used as the test substance. For the test, this substance was dissolved immediately before its use in absolute ethanol and was uniformly applied as a 10% solution (50 μl each) on the right dorsal epidermis of the ear using an automated pipette. The dorsal sides of the ear are suitable as test areas since these parts of the skin are almost without any hair. As controls, 50 μl absolute ethanol of the same batch was used in the area of the left ear.

As the UV source a HG high pressure radiation apparatus UVS 375-1 was employed which emits predominantly in the UV-B area. A group specific standardization of the UV-B erythema on the dorsal epidermis of the ear on the albino guinea pigs was carried out according to Höfer et al.: Zum zeitlichen Verlauf des UV-Erythems am Meerschweinchenohr, Vortrag: 4. Photodermatologisches Kolloquium mit internationaler Beteiligung, Wilten 10.-Oct. 12, 1988.

The dorsal parts of the ear without hair were irradiated with an irradiation power of.0.12 mW/cm²±10% (irradiation of the individual animal). The applied dose per animal was 0.108 J/cm² with the same tolerance. The dosimetric measurement which is decisive for the defined erythema production and reproduction was performed in analogy to Höfer et al., Dermatol. Mon.schr. 174 (1988) 87-93.

Measuring Methods and Exposure

The recording of the degree of inflammation was conducted using two different measuring methods being independent from each other, objective and are recording respective different inflammation symptoms.

As measuring principles, pyrometry (skin temperature) and reflection photometry (degree of skin reddening) were used (Gloor, Pharmakologie dermatologischer Externa, Springer Verlag Berlin Heidelberg New York, 1982, p. 134; Gloor et al., Dermatol. Mon. schr. 125 (1979), 665-669; Vane et al.: Anti-inflammatory Drug 5, Springer Verlag Berlin Heidelberg New York, 1979, p. 44-74 and Walter et al: lnfrarotmeβtechnik, VEB Verlag Technik Berlin, 1. Ed. 1981, p. 224). There was employed the digital hand pyrometer HPM 15 and a spekol 11 device (VEB Carl Zeiss, Jena) with a luminance measuring formulation R d/O.

50 μl of a 10% solution of 2-hydroxy-5-methyl-laurophenone oxime were uniformly applied to the animals in the dorsal area of the right ear 5 hours prior to the UV-B irradiation. The left side was treated with 50 μl of absolute ethanol as a control. In order to render the starting values objective, the animals were measured before this treatment. The room temperature during the whole duration of the experiment was between 18 and 21° C. The development of the erythema was measured 2, 4, 6, 7, 24, 48, 72 and 96 hours after erythema triggering in all animals at the same time of day.

Results

According to the present evidence, the development of a UV-B conditioned erythema on the ear of a guinea pig can be influenced in the sense of a suppression after a penetration time of 300 minutes of 50 μl of a 10% solution of 2-hydroxy-5-methyl-laurophenone oxime. This result was confirmed by both measuring principles. The phenomenon of the erythema suppression in comparison to the control is conspicuously distinct in particular during the early phase of the inflammation development. The results are shown in Table I and Table II.

TABLE I
Pyrometric characterization of the UV-B erythema on guinea pig
epidermis after the application of 50 μl of a 10% ethanol solution
of 2-hydroxy-5-methyl-laurophenone oxime (RHS ear) 5 hours prior to
triggering the erythema.
LHS ear = control

| | | | | | | | | |
|---|---|---|---|---|---|---|---|---|
| a | b | c | d | e | f | g | h | i |

Pyrometry [° C.]

| | | | | | | | | | |
|---|---|---|---|---|---|---|---|---|---|
| x (RHS) | 28.20 | 29.48 | 29.38 | 29.28 | 30.14 | 28.04 | 28.52 | 28.10 | 27.98 |
| ±s | 0.45 | 0.84 | 0.66 | 1.86 | 1.08 | 0.28 | 0.35 | 0.31 | 0.33 |
| x (LHS) | 27.30 | 31.46 | 31.92 | 32.50 | 33.44 | 28.34 | 28.66 | 28.60 | 28.10 |
| ±s | 0.50 | 0.49 | 0.34 | 2.13 | 1.31 | 0.21 | 0.36 | 0.25 | 0.33 |

T-test (significance threshold 2 P 0.005)

| | | | | | | | | |
|---|---|---|---|---|---|---|---|---|
| n.s. | s. | s. | n.s. | n.s. | n.s. | n.s. | n.s. | n.s. |

a = before treatment
b = 2 hours after treatment
c = 4 hours after treatment
d = 6 hours after treatment
e = 7 hours after treatment
f = 24 hours after treatment
g = 48 hours after treatment
h = 72 hours after treatment
i = 96 hours after treatment
x = mean value
±s = standard deviation
n.s. = not significant
s. = significant

TABLE II
Reflection photometric characterization of the UV-B-erythema on guinea
pig epidermis after the application of 50 μ1 of a 10% ethanol solution
of 2-hydroxy-5-methyl-laurophenone oxime (RHS ear) 5 hours prior
to triggering the erythema.
LHS ear = control

| | | | | | | | | |
|---|---|---|---|---|---|---|---|---|
| a | b | c | d | e | f | g | h | i |

Reflection Photometry [%]

| | | | | | | | | | |
|---|---|---|---|---|---|---|---|---|---|
| x (RHS) | 33.62 | 31.14 | 31.02 | 31.70 | 32.26 | 31.18 | 31.78 | 32.72 | 32.56 |
| ±s | 0.57 | 0.31 | 0.75 | 0.52 | 0.65 | 0.53 | 0.52 | 0.39 | 0.47 |
| x (LHS) | 33.16 | 28.52 | 28.34 | 27.88 | 29.66 | 30.96 | 32.18 | 32.58 | 32.44 |
| ±s | 0.80 | 0.50 | 1.20 | 1.37 | 0.78 | 0.50 | 0.49 | 0.48 | 0.68 |

T-test (significance threshold 2 p 0.005)

| | | | | | | | | |
|---|---|---|---|---|---|---|---|---|
| n.s. | s. | s. | n.s. | n.s. | n.s. | n.s. | n.s. | n.s. |

a = before treatment
b = 2 hours after treatment
c = 4 hours after treatment
d = 6 hours after treatment
e = 7 hours after treatment
f = 24 hours after treatment
g = 48 hours aftertreatment
h = 72 hours after treatment
i = 96 hours after treatment
x = mean value
s = standard deviation
n.s. = not significant
s. = significant

1. Use of at least one aryl oxime of Formula (I) 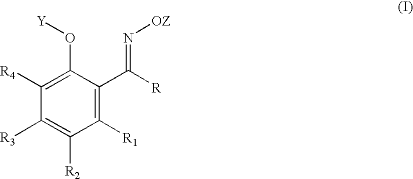
wherein:
Y, Z represent independently from each other H. C₁₋₁₈ alkyl, C₂₋₁₈ alkenyl, C₂₋₁₈ carboxy alkyl, C₃₋₁₈ carboxy alkenyl or C₂₋₁₈ alkanoyl;
R represents C₁₋₁₈ alkyl, C₂₋₁₈ alkenyl, C₃₋₈ cycloalkyl, aryl, aralkyl, heteroaryl, heteroaralkyl or condensed systems;
R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl,
for the prophylaxis and/or treatment of erythema formation and/or inflammation responses of the skin.

2. Use according to claim 1 for the prophylaxis and/or treatment of inflammation responses of the skin caused by physical or chemical noxae and/or foreign organisms.

3. Use according to claim 1 or 2 for the prophylaxis and/or treatment of inflammation responses of the skin caused by UV radiation.

4. Use according to claim 3 in the PUVA therapy.

5. Use according to one of claims 1 to 4 in form of a topical composition.

6. Use according to claim 5 characterized in that at least one aryl oxime of Formula (I) is present in a topical composition in an amount of 0.02 to 2 weight % based on the composition.

7. Use according to claim 5 or 6 characterized in that the topical composition further contains at least one antioxidant and/or at least one UV filter.

8. Use according to claim 7 characterized in that the antioxidant is present in the topical composition in an amount of 0.001 to 5 weight % based on the composition.

9. Use according to claim 7 characterized in that the UV filter is present in the topical composition in an amount of 0.01 to 30 weight % based on the composition.
